(19) United States
(12) Patent Application Publication
(10) Pub. No.: US 2010/0267017 A1
(43) Pub. Date: Oct. 21, 2010

(54) MONITORING REAL-TIME PCR WITH LABEL FREE INTRINSIC IMAGING

(76) Inventor: Stuart Hassard, Richmond (GB)

Correspondence Address: LANDO & ANASTASI, LLP, ONE MAIN STREET, SUITE 1100, CAMBRIDGE, MA, 02142, US

(21) Appl. No.: 12/095,699

(22) PCT Filed: Nov. 30, 2006
(86) PCT No.: PCT/GB06/50423

§ 371 Date: Jul. 6, 2010

(30) Foreign Application Priority Data
Nov. 30, 2005 (GB) 0524298.7

(51) Int. Cl. C12Q 1/68; C12M 1/34

(52) U.S. Cl. 435/6; 435/287.2

(57) ABSTRACT

The invention provides a method for the detection of nucleic acid the method comprising carrying out a PCR reaction in a microfluidic device wherein the sample shuttles within the microfluidic channel and the amount of nucleic acid is determined based on the UV absorption of the nucleic acid.

### BACKGROUND

The detection of nucleic acid and of nucleic acid modifications has many applications and is a particularly important tool in the diagnosis of disease. Most techniques employ the Polymerase Chain Reaction (PCR), which enables the amplification of very small amounts of complex genetic material.

The advent of PCR has hugely accelerated the progress of studies on the genetic structure of a diversity of organisms. PCR is an enzyme-catalysed reaction that allows any nucleic acid sequence to be generated in vitro, and in abundance. First reported in 1986, PCR has since become a requisite tool in basic molecular biology, genome sequencing, clinical research and evolutionary studies. The reason for the success of PCR lies in its simplicity. At high temperature (about 95° C.), the double-stranded target DNA denatures—unwinds into two single strands. Synthetic sequences of single-stranded DNA, known as primers, are used to bracket the region of the chain to be amplified: one primer is complementary to one DNA strand (at the start of the target region), with the second primer being complementary to the other DNA strand (at the end of the target region). The primers are annealed to the single strands when the local temperature is reduced to between 50 and 65° C. This is followed by ‘extension’, at a slightly higher temperature (about 72° C.), in which a complementary strand develops from each primer by the catalytic action of a DNA polymerase enzyme, in the presence of free deoxynucleotide triphosphates. This three-step process constitutes one PCR cycle, and if repeated n times will yield 2″ copies of the original DNA strand.

Conventional instruments for performing PCR (thermal cyclers) are conceptually simple, but possess a number of technical frailties that limit the speed and efficiency of amplification. A fundamental requirement for efficient amplification is rapid heat transfer. It is desirable to have a system with a low heat capacity that can transfer heat quickly to the sample on heating, and quickly away when cooling. Most conventional thermal cyclers have large thermal masses, resulting in high power requirements and protracted heating and cooling rates. Consequently, total reaction times are typically in excess of 90 minutes. As the denaturation and annealing steps occur as soon as the correct temperature is reached, and extension is limited only by the processing power of the polymerase enzyme (between 50 and 500 bases per second), total reaction times can be drastically shortened if the thermal mass of the instrument is reduced. In recent years, microfabricated PCR systems have been developed with this idea in mind: although diverse in structure, all rely on the reduction of thermal mass to facilitate rapid heating and cooling, and afford reaction times as short as a few minutes. The normal approach to instrument miniaturisation involves the direct downsizing of system dimensions to reduce thermal masses. Early studies used this concept to create microfabricated devices in which a static reaction chamber (with volumes between 100 pL and 50 μL) was thermally cycled using resistive heaters mounted externally or integrated within a monolithic substrate. Using this general approach significant improvement in reaction speed, analytical throughput and reaction efficiency have been demonstrated.

Traditional PCR methods analyse the product by agarose gel electrophoresis. The disadvantage is that this method is time consuming and shows low sensitivity. The basic PCR method has been developed further and methods are now available for detecting sequence-specific PCR products in real time. One such method is the TaqMan® assay (Holland. P. et al 1991. PNAS, 88 pp 7276-7280 Detection of specific polymerase chain reaction product by utilizing the 5′-->3′ exonuclease activity of Thermus aquaticus DNA polymerase.) wherein detection of PCR products is based on the detection of fluorescence of a reporter. However, despite its advantages, the TaqMan® assay also has some disadvantages. For example, sequence data for the construction of probes must be available. Therefore, the costs for the assay are particularly high when different probes need to be synthesised for the detection of different sequences. Another method for real-time PCR commonly used employs a dye (SYBR Green®, Lipsky, R. et al 2001 Clinical Chemistry 47 pp 635-644 DNA Melting Analysis for Detection of Single Nucleotide Polymorphisms), which binds specifically to double-stranded DNA, but not to single-stranded DNA. However, this method has the disadvantage that the dye is non-specific and can generate false positive signals. Other methods use molecular beacons or scorpions but similar to the TaqMan® assay, these methods are complex and expensive.

WO 03/102238 relates to a real-time PCR method by measuring UV absorbance of the PCR mixture. The contents of WO 03/102238 are hereby incorporated by reference. WO 03/036302 discloses a method for monitoring the folding and unfolding of proteins and an apparatus for analysing temperature-dependent configurations of proteins. The contents of WO 03/036302 are hereby also incorporated by reference.

Due to genetic variations, more than 90% of drugs provide effective treatment in only 30%-50% of a given population. In order to reduce this serious problem the healthcare industry is looking towards a more personal treatment regime. Currently, this age of personalised medicine is being delayed by the lack of diagnostic technology available at the Point-of-Care (PoC). Nucleic acid modifications include short tandem repeats (STR) and single nucleotide polymorphisms (SNPs). SNPs are DNA sequence variations that occur when a single nucleotide (A, T, C or G) in the genome sequence is altered. For a variation to be considered a SNP, it must occur in at least 1% of the population. Allele frequencies vary greatly, also amongst different populations. SNPs, which make up about 90% of all human genetic variation, occur every 100 to 300 bases along the 3-billion-base human genome. Because SNPs are usually only present in two forms, the allele that is more rare is referred to as mutant or minor allele and the most common allele is referred to as wild type allele. SNPs are primarily bi-allelic (i.e. there are two possible alleles at one locus) but may also be tri-allelic (i.e. two independent mutation events have occurred at the same time). Two of every three SNPs involve the replacement of cytosine (C) with thymine (T). SNPs can occur in both coding (gene) and non-coding regions of the genome (extronic or intronic).

Although more than 99% of human DNA sequences are the same across the population, variations in DNA sequence can have a major impact on how humans respond to disease, pathogens and therapies. This makes SNPs of great value for biomedical research and for developing pharmaceutical products or medical diagnostics. Therefore, the provision of an efficient, precise, cheap and user-friendly method for the detection of SNPs can be of great value. Current methods used to analyse SNPs include PCR followed by sequencing, microarrays and mass spectrometry. However, in particular microarrays and mass spectrometry are complex and expensive. One PCR method based on 3′ mismatch SNP scoring is exemplified by the GALIOS system (Weber, S. et al 2002 British Journal of Haematology 116 pp 839-843: Genotyping of human platelet antigen-1 by gene amplification and labeling in one system and automated fluorescence correlation spectroscopy). In this system two primers are used at the 5′ end of the putative product, one of which has a 3′ mismatch. The upper wild type template has a complementary primer, which the polymerase will be able to extend to the 3′. The lower SNP containing template cannot fully anneal the primer, creating a 3′ mismatch. This will result in markedly lower product, if indeed any. In GALIOS, and other related systems, product is detected by attached fluorescent dyes. Other labelled systems also exist such as Taqman® and SYBR green systems and their disadvantages are discussed above. Therefore, there is a need for an alternative and improved method for analysing SNPs.

Most DNA molecules show a relative increase of 1.4 in absorbance at 260 nm upon denaturation which is known as the hyperchromic effect. This effect arises due to the different structures of single (ssDNA) and double stranded DNA (dsDNA). ssDNA absorbs about 30% more photons at 260 nm than dsDNA. The increase in absorbance is caused by the exposure of the highly absorbing purine and pyrimidine rings, which in dsDNA are stacked internally to the helix, as they form the hydrogen bond moieties.

Microfluidic devices have been described elsewhere (Kopp et al, Science 280. 1998). For example, Münchow et al discloses a microfluidic PCR method wherein the PCR product is detected by fluorescence or gel electrophoresis (Münchow G et al 2005 Expert Rev. Mol. Diagn. 5 pp 613-620 Automated chip-based device for simple and fast nucleic acid amplification).

The present inventors have found an alternative method for nucleic acid detection which makes use of a semi continuous flow PCR and is based on label free intrinsic imaging, as no extrinsic label needs to be incorporated into the PCR product.

The invention also provides a method for allele specific primer, PCR based, SNP validation. Creation of such a method should allow a healthcare worker to take a blood sample from a patient and rapidly (within a few minutes) make an informed choice of drug therapy based on the patient's genetic information.

### SUMMARY OF THE INVENTION

In a first aspect, the invention relates to a method for the detection of nucleic acid the method comprising
a) amplifying a nucleic acid sample wherein a solution comprising a nucleic acid sample is moved along a temperature-controlled and UV illuminated channel and the flow direction of the solution is altered multiple times and
b) measuring UV absorption of the nucleic acid.

In another aspect, the invention relates to an apparatus for determining the presence of nucleic acid comprising at least one microchannel, a ferrofluidic actuation means, heating elements, a UV light source and a detector.

### DESCRIPTION OF THE INVENTION

According to a first aspect, the invention relates to a method for the detection of nucleic acid the method comprising
a) amplifying a nucleic acid sample wherein a solution comprising a nucleic acid sample is moved along a temperature-controlled and UV illuminated channel and the flow direction of the solution is altered multiple times and
b) measuring UV absorption of the nucleic acid.

Thus, according to the invention, the solution comprising the nucleic acid sample moves backwards and forwards within the channel. Therefore, the solution moves in a non-linear and non-continuous fashion. The sample shuttles between different parts of the channel.

The amplification is carried out while the solution shuttles. This is achieved by applying different temperatures to different parts of the channel.

The method allows determining the amount of nucleic acid present in the sample.

According to the invention, the term nucleic acid sample refers to a sample comprising DNA or RNA. The DNA may comprise cDNA and RNA may comprise mRNA or siRNA. Preferably, the nucleic acid sample comprises single-stranded DNA or RNA or double-stranded DNA or RNA. In one embodiment, the nucleic acid comprises native secondary structural elements or is in its denatured form. In one embodiment, the nucleic acid sample may comprise nucleic acid isolated from a microorganism, animal or plant. In another embodiment, the nucleic acid is a synthetic sequence, for example a part of a vector or an oligonucleotide. In a further embodiment, the nucleic acid sample comprises animal or plant cells or cells of a microorganism.

In one embodiment, the amplification is by PCR. According to the invention, PCR refers to a polymerase chain reaction for the amplification of DNA. Typically, a PCR reaction comprises the steps of denaturation, annealing and extension, which are carried out at different temperatures. Denaturation, the separation of two complementary strands, typically requires a standard temperature of about 95° C. The temperature required for annealing is dependent on the particular primer used but is typically carried out at about 54° C., but varies depending on the base composition of the primer. It may be between 40° C. and 60° C. Extension of primer molecules is typically carried out at about 72° C. A skilled person will appreciate that the temperatures used vary according to the type of PCR carried out and the primers used. PCR requires the presence of a polymerase enzyme to catalyse the reaction. Typically, DNA Pol I, Taq polymerase or any other thermally stable polymerase enzyme may be used.

PCR may be carried out in real time.

A primer is a short oligonucleotide which anneals to the complementary sequence within the target nucleic acid. Typically the primer is 15 to 30 nucleotides long. The sequence of oligonucleotide primers used in the reaction is dependent on the sequence of interest. If required, degenerate primers may be used in the method of the invention. The precise temperature control of the channel enables to accurately adjust the temperature required for the specific primer used.

In one embodiment method of the invention, the primer may also be labelled to provide a further level of detection. Such labels are known to the skilled person and include, fluorescent dyes or radioactive labels.

Thus, in one embodiment, the solution further comprises a PCR mixture. The term PCR mixture or PCR reagents according to the invention refers to a mixture comprising components typically required to perform a PCR reaction. Such mixture will typically comprise a buffer, a set of at least one oligonucleotide primer, dNTP's (Nucleotides consisting of the four DNA bases adenine (dATP), thymine (dTTP), guanine (dGTP) and cytosine (dCTP)), and a polymerase. It will be apparent to the skilled person that the components of the PCR mixture and their concentration and the conditions used may be varied according to the type of PCR reaction performed.

The skilled person will be aware that the method according to the invention may relate to different types of PCR reactions, such as hot start PCR, inverse PCR, RT-PCR, RACE, nested PCR, asymmetric PCR and other PCR methods. Thus the invention also relates to a method according to the invention wherein hot start PCR, inverse PCR, RT PCR, RACE, nested PCR, asymmetric PCR is carried out.

The term temperature-controlled according to the invention refers to controlling the temperature along the length of the channel, in other words, a temperature profile is applied to the length of the channel and thus to the solution within the channel. According to the invention, the temperature is controlled so that a range of temperatures can be applied to the solution along the length of the channel. Thus, a specified temperature can be applied to the channel (and thus the solution) at any given poirit along the length of a channel. Thus, it is possible to apply a specific temperature to the solution at each possible position along the length of the channel. The channel may comprises Peltier cells as temperature controlling elements. Temperature resolution can be adjusted to suit experimental conditions, but resolution of 1° C. per millimetre or lower may be used. In one embodiment, the channel is a microchannel, for example on a chip, such as described in Kopp et al, Science 280, 1998.

In one embodiment, the temperature of the channel is within the range of 40° C. to 110° C.

In another embodiment, different temperatures zones are applied to separate parts of the channel. Preferably, the temperature in the first temperature zone is within the range of 40° C. to 60° C., in the second temperature zone about 72° C. and in the third temperature zone about 95° C.

According to the invention, amplification is achieved by shunting a solution comprising a DNA sample and PCR reagents (“sample plug”) back and forth, over static heating zones, in a microfluidic channel, for example by applying alternate left-right pressure as shown in FIG. 2 (Münchow et al). The method combines the cycling flexibility of static or well-based reactors with the rapid temperature transitions (and ultra-low thermal masses) associated with continuous-flow PCR microstructure. In contrast to continuous flow PCR using microfluidic methods known in the art, the method of the invention thus provides a semi-continuous flow PCR combined with the detection of nucleic acid on the basis of measuring UV absorption of the nucleic acid.

According to the methods of the invention, information on the efficiency of PCR is acquired from data relating to the hyperchromic effect (FIG. 4). Thus, UV absorption of the nucleic acid sample is measured at about 260 nm and the production of nucleic acid during the amplification can be monitored.

Imaging the product plug during the denaturation phase at 95° C. may be enhanced by this feature. It may be envisioned that the increase in dsDNA product will not be represented by a smooth exponential curve, rather it is expected to have features related to the hyperchromic effect, resulting in extra absorbance generated as the DNA denatures. FIG. 5 shows a representation of how the product curve may appear.

A series of step features will be generated as the amount of product increases. This new dsDNA product in the 72° C. zone will increase the absorbance at 260 nm as it is created. As the reaction plug shuttles to the 95° C. zone it will melt to ssDNA and show a signal increase due to both the amount of product and the hyperchromic effect. As it shuttles back to the 72° C. there will be a small initial drop in absorbance due to the re-naturing of the strands, however this will soon be compensated for by the increase in dsDNA. This is one of a number of possible outcomes. It may be that no step features will occur, or that the increase at 95° C. will produce a much sharper angle of product curve. It is also important to consider that the information gained will be dependent on the speed of data sampling. Imaging systems may operate at frequencies of 20 Hertz (Hz) and higher across the 512 pixels of the Photo Diode array or elements of a Charged Coupled Device (CCD). However, as the clock speed used is much higher and consequently ultrafast (10 s of microsecond), imaging is also possible and can be made to match the system chemistry dynamics. Multiple detectors may be used, and the speed of the shuttling plug controlled very accurately so that the system will be tunable and can acquire the best data possible within the denaturing zone.

The concentration or amount of the PCR product is monitored based on measuring UV absorption. Concentration is detected by causing the nucleic acid to pass between a light source and a light detector. For example, by using UV sensitised Photo Diode Arrays (PDAs) or charge-coupled devices (CCDs) as detectors, the speed at which the DNA band moves across the channel can be determined, thus giving a measure of the plug length of the sample.

In one embodiment, the method comprises measuring the velocity of the nucleic acid in the sample. The velocity of the molecule can be established by the use of multiple detection as the molecule traverses one or more photo diode arrays of 512 or more pixels. This allows a space-time correlation to be established. The position of the molecule within the channel is detected based on the UV absorption of the nucleic acid. The molecules are illuminated by Ultra Violet light from a deuterium lamp or UV diode laser which they maximally absorb at 260 nm, causing a drop in signal at the pixel of the PDA detector they are traversing. Thus, the nucleic acid may be identified by signal which can be used to obtain a measure of the amount present. It is also possible to calculate the velocity that is needed for the sample to reach a predetermined position and based on the velocity of the sample, the amount present can be determined. The techniques used according to the invention for velocity based signal processing are described in WO96/35946, WO 02/12876 and WO 02/12877. Multipixel detection also yields an increase in single-to-noise ratio when appropriate space-time averaging is performed. Placing a PDA close to the ends of the channel, and close to the temperature zones, will allow monitoring of any temperature dependent features exhibited by the DNA. The exact configuration of the detectors, their number and their position will be a function of the imaging constraints introduced by the speed and size of the PCR plug. The sample plugs is positioned at t=0 before it begins to move. This allows the application of velocity based signal processing. In the proposed system t=0 will be relatively easy to establish as the plug will be stationary in the system as a function of the temperature zone it is in. In one configuration, the detectors may be positioned contiguous to the heating element, or orthogonally.

Thus, in one embodiment, the method of the invention comprises velocity based signal processing.

One of the advantages of the proposed method is that it allows real-time measurements to be performed. The number of PCR cycles needed for satisfactory amplification using conventional PCR techniques is at least 30. The method of the invention can significantly reduce the numbers of cycles need to produce a detectable amount of product. As few as 10 cycles of a typical 300 bp PCR product have been imaged using LFII technology (FIG. 7). According to the invention, PCR is carried out using 10 to 40, preferably 20 cycles. The amount of times that the flow direction of the solution comprising the sample and the PCR mixture, in other words the PCR sample plug, is altered thus depends on the cycles of the PCR used.

Furthermore, even with the best of the PCR machines in general use, a PCR reaction will take at least an hour. More expensive machines can do it in less time, but they are not in general laboratory use. According to the method of the invention, the reaction time can be reduced significantly.

Furthermore, the method requires low sample and reagent volumes typically, but not limited to the nL range. Due to the low instantaneous volumes and associated thermal masses, sample plugs will thermally equilibrate on a time scale of some between 10 and 100 ms when transported to a different temperature zone. Consequently, the thermal limitations on the cycle speed of the system are greatly reduced when compared with both macro- and microfluidic batch cyclers. Moreover, since PCR reagents will be contained within a relatively short microchannel (when compared with a continuous-flow system) there is less adsorption of vital reaction components such as the DNA polymerase) onto channel surfaces.

The microfluidic channel used in the methods of the invention may be made using current state of the art microfluidic techniques such as SU-8 photolithograghy, and polydimethylsilaxane (PDMS), TOPAS or other UV transparent plastic chip microfabrication, laser or machine tool or wet etching of a plurality of UV transparent glasses or quartz materials.

The method may use either pressure or ferro-fluidic actuation to manipulate the flow direction of the solution within the channel. In the case of ferro-fluidic actuation, the solution further comprises oil and magnetic nanoparticles. A pressure of 100 mbar may be applied. Although the implementation and integration of pressure-actuators is facile, magnetic manipulation of a ferro-fluid plug is likely to provide for the optimum control of sample plug motion and reagent diffusion if performed in a looped microfluidic systems as shown in FIG. 2. Using either approach we expect that the sample plug can be driven through between 20 and 60 thermal cycles within a period of five minutes.

Label free intrinsic imaging according to the invention allows real time detection of the product without the need for the inclusion of additional labels. Furthermore, using detectors, the amount of product can also be quantified according to the invention.

Preferably, the methods of the invention can be carried out so that a device with a plurality of microchannels is used.

In another aspect, the invention provides the use of the method for determining the presence of a nucleotide modification. A nucleotide modification may be the substitution, deletion or addition of a nucleotide or base pair. According to the invention, one or more nucleotide modifications may be detected. In particular, the modification may be STR, SNP, a Targeted Genetic Modification (GM) step. Preferably; the nucleotide modification is a SNP. In this system two primers are used at the 5′ end of the putative product, one of which has a 3′ mismatch, as shown in FIG. 4. The upper wild type template has a complementary primer, which the polymerase will be able to extend to the 3′. The lower SNP containing template cannot fully anneal the primer, creating a 3′ mismatch. This will result in markedly lower product, if indeed any. The product is detected by Label Free Intrinsic Imaging as described herein.

A possible instrumentation layout for carrying out the invention is shown in FIG. 1. The light source (a Deuterium lamp—HEREAUS Noblelamp DS 225/05J, optical parts (UV lenses-Newport, UV filters-Andover Optic), separation phase (Capillaries-Composite Metal Services Ltd) and detector (HAMAMATSU PDA 3904 S3904-512Q) are arrayed on a common rail. Light from the low-noise deuterium lamp or passes through a filter wheel allowing the selection of detection wavelength. The light is then focused on a fused silica capillary, typically with an internal diameter of 50-100 micrometers (μm). As a biomolecule passes the light beam it absorbs energy dependent on its spectral characteristics. The light beam is then focused on to the detector where the drop in signal due to the energy absorbed by the biomolecule is measured.

Accordingly, in a further embodiment, the invention provides an apparatus for determining the presence of nucleic acid comprising at least one microchannel, a ferrofluidic actuation means, heating elements, a UV light source and a detector.

The detector is preferably a photo diode array or a charge coupled device.

In one embodiment, the apparatus comprises a plurality of parallel channels which are imaged simultaneously, using an array of CCDs.

It should also be noted that the microfluidic devices can and will be integrated with both upstream and downstream processing Components, such as DNA extraction and product sizing. Consequently, the microfluidic platform will be used to perform all processing tasks between sample extraction (from the patient) to final SNP validation.

### DESCRIPTION OF THE FIGURES

FIG. 1 illustrates the instrumentation layout.

FIG. 2 shows a multichannel semi continuous PCR method.

FIG. 3 illustrates how a sample plug is moved repeatedly between two controlled heating zones to effect strand denaturation, annealing and extension in each cycle of a PCR reaction.

FIG. 4 illustrates PCR based SNP analysis.

FIG. 5 illustrates the hyperchromic effect of DNA.

FIG. 6 shows a possible product curve.

FIG. 7 shows the product from 10 PCR cycles for 300 bp denatured DNA fragment (separation in 5MDa (2.5% conc) PEO; 40 cm separation length). The top panel show single pixel data, the bottom one shows processed data using the signal processing algorithms.

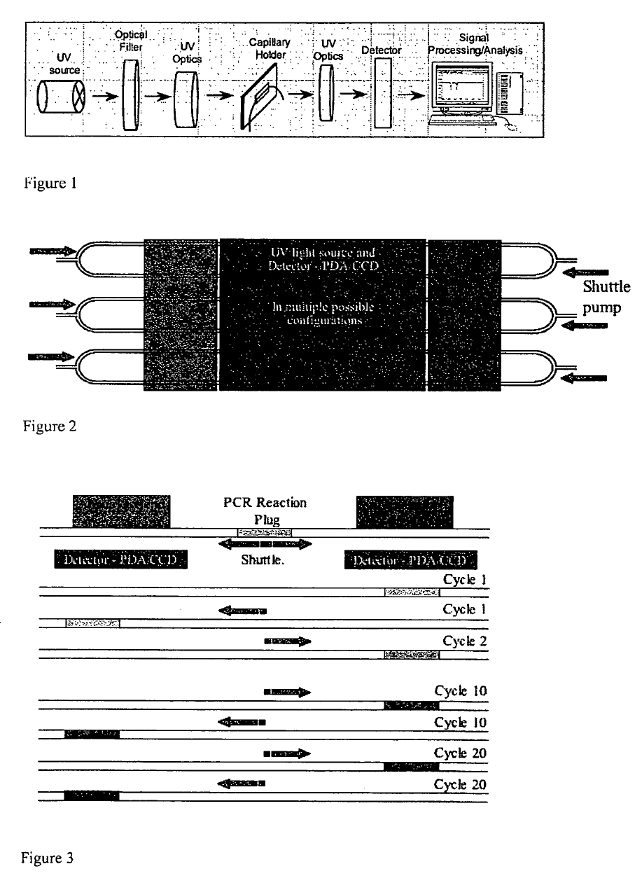

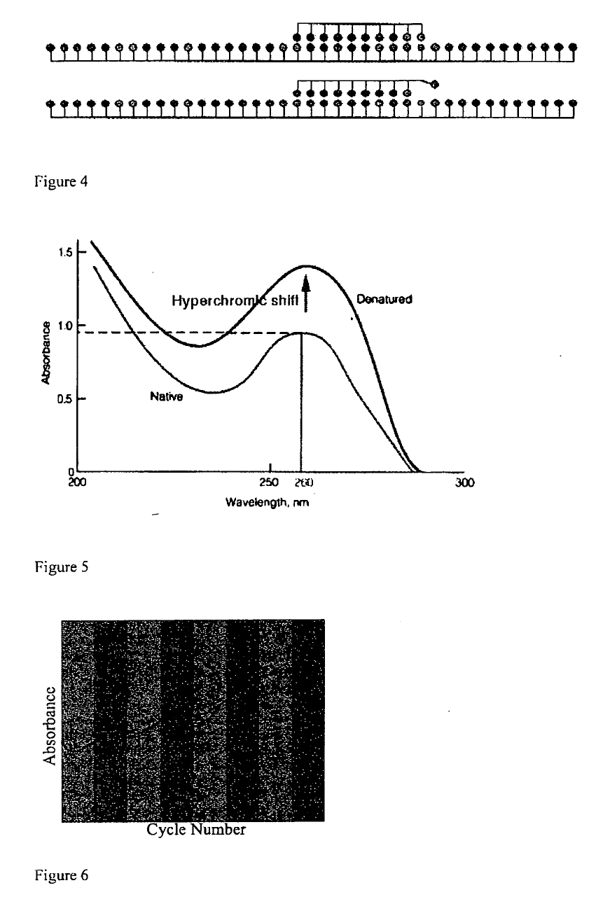

1. A method for the detection of nucleic acid the method comprising
a) amplifying a nucleic acid sample wherein a solution comprising a nucleic acid sample is moved along a temperature-controlled and UV illuminated channel and the flow direction of the solution is altered multiple times and
b) measuring UV absorption of the nucleic acid.

2. The method of claim 1 further comprising measuring the velocity of the nucleic acid.

3. The method of claim 2 using velocity based signal processing.

4. The method of any preceding claim comprising determining the amount of nucleic acid present.

5. The method of any preceding claim wherein the nucleic acid is DNA or RNA.

6. The method of any preceding claim wherein amplification is by PCR.

7. The method of any preceding claim wherein the solution further comprises a PCR mixture.

8. The method of claim 7 wherein the PCR mixture comprises at least one complementary primer and a polymerase.

9. The method of claim 8 wherein the primer is not labelled.

10. The method of claim 8 wherein the primer further comprises a label.

11. The method of claim 10 wherein the label is fluorescence.

12. The method of any preceding claim wherein the temperature of the channel is within the range of 50° C. to 100° C.

13. The method of any preceding claim wherein three different temperatures zones are applied to separate parts of the channel.

14. The method of claim 13 wherein the temperature in the first temperature zone is within the range of 50° C. to 60° C., the temperature in the second temperature zone is about 72° C. and in the third temperature zone about 95° C.

15. The method of any preceding claim wherein the number of PCR cycles is 10 to 40.

16. The method of claim 15 wherein the number of PCR cycles is 20.

17. The method of any preceding claim wherein the amount of nucleic acid is represented by the detected absorption of UV light by the nucleic acid molecules.

18. The method of claim wherein the amount of nucleic acid produced in a PCR cycle is represented by the detected absorption of UV light by the nucleic acid molecules.

19. The method of any preceding claim wherein UV absorption is detected using a photo diode array or a charge coupled device.

20. The method of any preceding claim wherein the solution is moved using pressure or ferrofluidic actuation.

21. The use of the method of any of claims 1 to 20 for the detection of a nucleic acid modification.

22. The use of claim 21 wherein the nucleic acid modification is SNP.

23. An apparatus for determining the presence of nucleic acid comprising at least one microchannel, a ferrofluidic actuation means, heating elements, a UV light source and a detector.
